(19) United States
(12) Patent Application Publication
(10) Pub. No.: US 2015/0182451 A1
(43) Pub. Date: Jul. 2, 2015

(54) CLEANSING COMPOSITION FOR HORMONE DEPOSITION

(71) Applicant: The Dial Corporation, Scottsdale, AZ (US)

(72) Inventors: Chris Luciow, Phoenix, AZ (US); Aurora Stamper, Phoenix, AZ (US); Mary J. Conway, Phoenix, AZ (US)

(21) Appl. No.: 14/141,647

(22) Filed: Dec. 27, 2013

(51) Int. Cl. A61K 9/00; A61K 47/14; A61K 38/27; A61K 47/10; A61K 31/568; A61K 31/565

(52) U.S. Cl. CPC A61K 9/0014; A61K 31/568; A61K 31/565; A61K 38/27; A61K 47/10; A61K 47/14

(57) ABSTRACT

Materials and apparatus are provided for hormone deposition in a cleansing composition. A cleansing composition includes a number of surfactants disposed within the cleansing composition. The cleansing composition also includes a dermal absorption enhancer disposed within the cleansing composition configured to deposit a hormone onto a skin surface. The cleansing composition also includes a number of hormones disposed within the cleansing composition, in which the number of hormones are configured to be deposited on a skin surface.

### FIELD OF THE INVENTION

The present invention generally relates to hormone deposition, and more particularly relates to a cleansing composition for hormone deposition.

### BACKGROUND OF THE INVENTION

Hormones may produce a number of desired effects in an individual. For example, with an aging population, there may be a need for new products to alleviate the signs and symptoms of aging. For example, the hormones may provide many benefits including improved feeling of vitality, sexual drive, mental capacity, weight loss among other benefits. A number of products may deliver hormones to the body.

Accordingly, it is desirable to deliver a determined amount of a hormone to achieve a desired function. Additionally, it is desirable to deliver the hormone in a fashion that is both safe for the consumer, and simple to implement. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A cleansing composition for hormone deposition includes a number of surfactants disposed within the cleansing composition. The cleansing composition also includes a dermal absorption enhancer disposed within the cleansing composition to deposit a hormone onto a skin surface. The cleansing composition also includes a number of hormones disposed within the cleansing composition, in which the number of hormones are configured to be deposited on the skin surface.

A cleaning product for depositing hormones on a skin surface includes an aqueous medium. The aqueous medium further comprises a cleaning component. The cleansing component includes at least one surfactant, a hormone configured to be deposited on the skin surface and a dermal absorption enhancer to deposit the hormone.

A cleansing composition for hormone deposition includes a primary surfactant forming 1.0 to 50.0 weight percent of the cleansing composition. The cleansing composition also includes a secondary surfactant forming 1.0 to 30.0 weight percent of the cleansing composition. The cleansing composition also includes a dermal absorption enhancer forming 0.10 to 2.0 weight percent of the cleansing composition. The cleansing composition also includes a hormone forming 0.0001 to 50.0 weight percent of the cleansing composition. The cleansing composition also includes a skin conditioning ingredient forming 0.1 to 2.0 weight percent of the cleansing composition.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

As described above, hormones may be used to carry out specific targeted functions within an individual. For example, testosterone may be delivered to a male individual to effect a physiological change and estrogen may be delivered to a female individual to effect a physiological change. These hormones may effectuate any number of desired functions. For example, a hormone may improve a feeling of vitality, sexual drive, mental capacity, and weight loss, among other physiological results. Hormones may be delivered to the individual in a number of ways. For example, they may be delivered orally. In another example, hormones may be absorbed through the skin via a hormone patch, or a leave-on product that is applied to the surface of the skin.

While such hormone delivery products may be beneficial, some characteristics of the hormone delivery products may result in a less than satisfactory consumer experience and may also pose safety concerns. For example, a hormone patch or a leave-on hormone delivery product, may deliver too much of an active hormone. Moreover, the hormone patch or leave-on hormone delivery product may leave behind a residue on the skin which may be uncomfortable, unsightly, may be an irritant to the skin, or may pose a safety concern for the consumer or other individuals who come in contact with the hormone-exposed skin.

Accordingly, the principles described herein provide a cleansing composition that may include hormones to carry out specific functions. More specifically, the principles described herein may use a cleansing composition, applied to the scalp or the body, to deliver a hormone to an individual via the skin. The cleansing formulation may also include surfactants, skin conditions, and dermal absorption enhancers to enhance the deposition of the hormone on the skin. Such a cleansing composition may be a rinse-off composition.

Accordingly, the cleansing formulation that deposits the hormone may be beneficial in that it is safe for consumers, delivers a correct dose of a hormone, and reduces potential safety concerns otherwise associated with hormone deposition.

As used in the present specification and in the appended claims, the term “a number of” or similar language may include any positive number comprising 1 to infinity; zero not being a number, but the absence of a number.

The cleansing composition may be included in a cleaning product that is configured to deposit a hormone to a scalp, such as in a shampoo. In another example, the cleansing composition may be included in a cleaning product that is configured to deposit a hormone on another skin surface, such as in a body wash. Another example of a cleaning product that may be configured to deposit a hormone on a skin surface is liquid hand soap. Accordingly, the cleansing composition may be included in a liquid hand soap. In yet another example, the cleansing composition may be included in a cleaning product that is configured to deposit a hormone on the face, such as a face wash.

In some examples, the cleansing composition may be held in a container that has an opening that allows the cleansing composition to flow out of the container onto a skin surface. The cleansing composition may be deposited on any skin surface including hands, arms, and scalp, among other skin surfaces. In some examples, the cleansing composition may be a rinse-off composition. In other words, the cleansing composition may be applied to the scalp, body or other skin surface and may deposit the hormones onto the skin while washing the skin. Water may be used to wash off excess cleansing composition. In some examples, the cleansing composition may be a prescription composition. In other words, the cleansing composition may deposit a prescribed amount of hormone to a skin surface. In other examples, the cleansing composition may be an over-the-counter cleansing composition. As used in the present specification and in the appended claims, the phrase “over-the-counter” may refer to cleansing compositions that are available to the public without a doctor's prescription.

The cleansing composition may include a number of surfactants disposed within the cleansing composition. The surfactant may further assist in hormone deposition by lowering the surface tension of a liquid and a solid. When added to the cleansing composition , a surfactant significantly reduces the surface tension of the water allowing the cleansing composition to remain on a skin surface for a longer period of time. Accordingly, the cleansing composition may include any surfactant that reduces the surface tension of water on a skin surface.

Surfactants have a hydrophobic end and a hydrophilic end. The hydrophobic end has an uncharged carbohydrate group that can be straight, branched, cyclic or aromatic. Depending on the nature of the hydrophilic part, the surfactants are classified as anionic, nonionic, cationic or amphoteric. Anionic surfactants have a hydrophilic end that has a negatively charged group like a sulfonate, sulfate, or carboxylate and are sensitive to water hardness. Nonionic surfactants include a non-charged hydrophilic part, e.g. an ethoxylate. Nonionic surfactants are not sensitive to water hardness. Cationic surfactants have a hydrophilic end that contains a positively-charged ion. Amphoteric surfactants or Zwitterionic surfactants have both cationic and anionic centers attached to the same molecule. The surfactants in the cleansing composition may include any appropriate type of mixture of surfactants. For example, the surfactants may include a blend of anionic and nonionic surfactants. The cleansing composition may include a primary surfactant forming between 1.0% and 50.0% weight of the cleansing composition, such as 5.0% and 10.0% weight. In some examples, the primary surfactant may be an alkyl ether sulfate, an alkyl sulfate, an isothionate, a sulfonate or combinations thereof. Alkyl ether sulfates are salts of the sulfuric acid monoesters of alkoxylated alcohols and may have the structure R(OCH₂CH₂)ₙOSO₃⁻M⁺. In this notation, “R” may represent an aliphatic fatty alkyl chain. The letter “n” may represent the degree of ethoxylation. The “M⁺” may indicate an alkali metal or amine.

A non-exhaustive list of examples of primary surfactants include, Sodium Laureth Sulfate, Ammonium Laureth Sulfate, Sodium Lauryl Sulfate, Sodium C14-16 Olefin Sulfonate, Sodium Coco-Sulfate, or combinations thereof. While specific reference is made to specific surfactants, any number of surfactants may be implemented in accordance with the principles described herein.

In some examples, the cleansing composition may include a secondary surfactant forming between 1.0% and 30.0% weight of the cleansing composition, such as 2.0% to 5.0%. A secondary surfactant may enhance the ability of the primary surfactant to reduce the surface tension of the cleansing composition. In some examples, the secondary surfactant may be a betaine, sultane, sulfosuccinate, amine oxide, amphoacetate, or combinations thereof. A betaine may include a quaternized alkyl or substituted alkyl derivative of N,N dimethyl glycine. A betaine may be a zwitterionic, surface-active compound. A non-exhaustive list of examples of secondary surfactants include Cocamidopropyl Betaine, Disodium Laureth Sulfosuccinate, Lauramine or Cocamine Oxide, Sodium Cocoamphoacetate, Decyl Glucoside, or combinations thereof. While specific reference is made to specific secondary surfactants, any number of surfactants may be implemented in accordance with the principles described herein.

The cleansing composition may include a number of hormones disposed within the cleansing composition. The number of hormones may be configured to be deposited on a skin surface. Specific examples of hormones that may be deposited on the skin surface include testosterone, estrogen, human growth hormone, or combinations thereof. While specific reference is made to testosterone, estrogen, and human growth hormone, any number of hormones may be included in the cleansing composition. For example, hormones may be included in the cleansing composition to carry out any number of physiological effects. For example, hormones may be sued to increase a feeling of vitality, increase a sexual drive, improve mental capacity, reduce the effects of aging, and enhance weight loss among other physiological effects. Accordingly, the cleansing composition may include any number of hormones to target any number of physiological conditions. The number of hormones may form between 0.0001% to 50.0% weight of the cleansing composition, such as 0.0001% to 0.5% weight. Including a number of hormones in the cleansing composition may be beneficial in that the hormones may be delivered to a consumer via a simple and effective cleansing composition that may avoid an uncomfortable and potentially dangerous residue on the skin surface.

The cleansing composition may include a dermal absorption enhancer disposed within the cleansing composition to deposit a hormone onto the skin surface. As described above, in some examples, the cleansing composition may be a rinse-off composition. In this example, the dermal absorption enhancer may facilitate the deposition of the hormone on the skin surface even though the cleansing composition may eventually be washed off by water. The dermal absorption enhancer may be configured to deliver a determined amount of hormone to the skin surface. For example, the dermal absorption enhancer may be configured to facilitate absorption of a prescribed amount of hormone into the skin surface. The dermal absorption enhancer may facilitate absorption by including a compound that helps to penetrate a lipid barrier in the stratum corneum. The dermal absorption enhancer may utilize a number of absorption processes to aid in the absorption of the hormone. For example, the dermal absorption enhancer may include ethanol which may carry the hormone through outer layers of a skin surface. An ethanol dermal absorption enhancer may work by removing a skin lipid barrier from the stratum corneum layer of the skin. Doing so may make the skin more permeable. Accordingly, the ethanol may enhance hormone deposition through the skin by making the skin more permeable and enhancing penetration of the hormone through the skin.

Other examples of dermal absorption enhancers include hydrophobically modified polymers, and other modified polymers that aid in the absorption of the hormone across the layers of the skin surface. Polymers may aid in dermal absorption of hormones by helping the hormones bind to the skin. Accordingly, a hormone has more opportunity to diffuse through a skin layer. Examples of polymers that may be used as polymers include quaternary ammonium compounds that include salts. The quaternary ammonium compounds may refer to positively charged tetra-substituted nitrogen derivatives. Accordingly, a polymer dermal absorption enhancer may include any positively charged tetra-substituted nitrogen. A non-exhaustive list of polymer dermal absorption enhancers may include Polyquaternium-10, Polyquarternium-7, Polyquaternium-39, Hydroxypropyl Guar Hydroxyprolyltrimonium Chloride or combinations thereof. While specific reference is made to specific polymer dermal absorption enhancers, any number of polymers may be implemented in accordance with the principles described herein.

In some examples, the dermal absorption enhancer may implement charged molecules to aid in carrying the hormone across a negatively charged skin surface. A charged molecule may bind the hormone to the skin. Accordingly, a hormone has more opportunity to diffuse through a skin layer. The dermal absorption enhancer may be an antistatic agent that alters the electrical properties of other compounds or skin surfaces by reducing their tendency to acquire an electrical charge. Accordingly, an antistatic agent may enhance dermal absorption by modifying the skin surface's characteristics.

A non-exhaustive list of antistatic agents that may be used as dermal absorption enhancers may include Cocamidopropyl PG-Dimonium Chloride, Cocamidopropyl PG-Dimonium Chloride Phosphate, Sunfloweramidopropyl Ethyldimonium Ethosulfate, Isostearamidopropyl Morpholine Lactate, or combinations thereof. While specific reference is made to specific antistatic agents, any number of antistatic agents may be implemented in accordance with the principles described herein.

In another example, the dermal absorption enhancer may be a solvent. A solvent may be a liquid employed to dissolve components. A solvent dermal absorption enhancer may work by removing a skin lipid barrier from the stratum corneum layer of the skin. Doing so may make the skin more permeable. Accordingly, the solvent may enhance hormone deposition through the skin by making the skin more permeable and enhancing penetration of the hormone through the skin.

A non-exhaustive list of solvents that may be used as dermal absorption enhancers may include Denatured Alcohol, Ethanol, Isopropyl Alcohol, Ethyl Lactate, PEG-8, PEG-10, PEG-12, PEG-6 Methyl Ether or combinations thereof. While specific reference is made to specific solvents, any number of solvents may be implemented in accordance with the principles described herein.

In yet another example, the dermal absorption enhancer may be an encapsulant that encapsulates the hormone. Upon deposition on a skin surface, the encapsulant may break down to release the hormone.

The dermal absorption enhancer may be configured to deposit the hormone at different layers of the skin surface. For example, the dermal absorption enhancer may be configured to deposit the hormone at the stratum corneum layer of a skin surface among other skin surface layers. In another example, the dermal absorption enhancer may be configured to carry the hormone deeper, to the blood stream, for example. In some examples, the dermal absorption enhancer may form 0.10% to 2.0% weight of the cleansing composition.

Including the dermal absorption enhancer in the cleansing composition may be beneficial in that a predetermined amount of the hormone may be delivered. For example, as described above, leave-on products and hormone patches may deliver too much hormone. Accordingly, the dermal absorption enhancer may ensure that a desired dose is administered, and that an overdose is avoided. More particularly, the dermal absorption enhancer may ensure that a correct dose is administered, notwithstanding the use of a rinse-off cleansing composition may be washed away with water.

In some examples, the cleansing composition may include a skin conditioning ingredient to enhance the deposition of the hormone on the skin surface. In general, skin conditioning ingredients prepare the skin for deposition of a hormone and for increased absorption of the hormone. For example, a skin conditioning ingredient may increase water content within the stratum corneum and may maintain the suppleness of the skin. In some examples, the skin conditioning ingredient may form 0.1% to 2.0% weight of the cleansing composition.

The cleansing composition may also include other ingredients to increase the “feel” and aesthetic qualities of the cleansing composition. Such ingredients may include a dye, a fragrance, a preservative, a PH adjuster, an antiseptic, a humectant, a thickener, a humectant, a conditioning agent, a chelating agent, a viscosity adjuster, a foam booster among other aesthetic enhancing ingredients. The cleansing composition may also include an aqueous medium, such as water, to contain the surfactant, number of hormones, and dermal absorption enhancer.

Table (1) gives an exemplary list of components of a cleansing composition with corresponding weight percentages of the cleansing composition.

TABLE (1)

| Component | Weight Percentage |
|---|---|
| Primary Surfactant | 1%-50% |
| Secondary Surfactant | 1%-30% |
| Fragrance | 1.0% |
| Dermal Absorption Enhancer | 0.10%-2.0% |
| Hormone | 0.0001%-50.0% |
| Skin Conditioning Ingredient | 0.1%-2.0% |

As indicated above, the cleansing composition described above may be beneficial in that may delivered a correct desired dosage of hormone to an individual. Moreover, the cleansing composition may avoid a residue that may be uncomfortable for a user and dangerous to contact from any individual. The cleansing composition may also show proof of enhanced absorption in a rinse-off composition and may meet other consumer performance attributes such as lather and skin feel among other aesthetic qualities. These benefits, in addition to others, are provided by the cleansing composition as described herein.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1. A cleansing composition for hormone deposition, comprising:
at least one anionic surfactant disposed at a concentration ranging between 5.0 and 50.0 weight % within the cleansing composition and selected from the group consisting of alkyl ether sulfates, alkyl sulfates, and alkyl sulfonates;
a dermal absorption enhancer disposed within the cleansing composition to deposit a hormone onto a skin surface and comprising a positively charged quaternary ammonium compound or salt thereof; and
at least one hormone disposed within the cleansing composition, in which the at least one hormone is configured to be deposited on the skin surface.

2. The cleansing composition of claim 1, in which the at least one hormone is selected from the group consisting of testosterone, estrogen, and human growth hormone.

3. The cleansing composition of claim 1, in which the at least one hormone form 0.0001 to 50.0 weight percent of the cleansing composition.

4. The cleansing composition of claim 1, further comprising a dye, a fragrance, a thickener, or combinations thereof.

5. The cleansing composition of claim 1, in which the dermal absorption enhancer is further comprises one or more members selected from the group consisting of a solvent, a polymer, an encapsulant, and an additional charged molecule.

6. The cleansing composition of claim 1, in which the dermal absorption enhancer is configured to deposit the hormone on an epidermal layer of skin.

7. The cleansing composition of claim 1, in which the dermal absorption enhancer is further configured to carry the hormone to the blood stream.

8. The cleansing composition of claim 1, further comprising a skin conditioning ingredient to enhance the deposition of the hormone on the skin.

9. The cleansing composition of claim 8, in which the skin conditioning ingredient forms 0.1 to 2.0 weight percent of the cleansing composition.

10. The cleansing composition of claim 1, further comprising a secondary surfactant.

11. The cleansing composition of claim 1, in which the cleansing composition is a rinse-off composition.

12. A cleansing product for depositing hormones on a skin surface, comprising:
an aqueous medium; and
the aqueous medium further comprising a cleansing component comprising:
at least one anionic surfactant at a concentration ranging between 5.0 and 50.0 weight % of the cleaning product and selected from the group consisting of alkyl ether sulfates, alkyl sulfates, and alkyl sulfonates;
a hormone configured to be deposited on the skin surface; and
a dermal absorption enhancer to deposit the hormone and comprising a positively charged quaternary ammonium compound or salt thereof.

13. The cleansing product of claim 12, in which the cleansing composition is a rinse-off composition.

14. The cleansing product of claim 12, in which the cleansing composition is configured to be used as a shampoo.

15. The cleansing product of claim 12, in which the cleansing composition is configured to be used as a body wash.

16. The cleansing product of claim 12, in which the cleansing composition is a prescription cleansing composition.

17. The cleansing product of claim 12, in which the cleansing composition is an over-the-counter cleansing composition.

18. A cleansing composition for hormone deposition, comprising:
a primary anionic surfactant forming 5.0 to 50.0 weight percent of the cleansing composition and selected from the group consisting of alkyl ether sulfates, alkyl sulfates, and alkyl sulfonates;
a secondary surfactant forming 1.0 to 30.0 weight percent of the cleansing composition;
a dermal absorption enhancer forming 0.10 to 2.0 weight percent of the cleansing composition and comprising a positively charged quaternary ammonium compound or salt thereof;
a hormone forming 0.0001 to 50.0 weight percent of the cleansing composition; and
a skin conditioning ingredient forming 0.1 to 2.0 weight percent of the cleansing composition.

19. The cleansing composition of claim 18, in which the hormone is at least one member selected from the group consisting of testosterone, estrogen, and human growth hormone.

20. The cleansing composition of claim 18, in which the cleansing composition is a rinse-off composition.
